(19) United States
(12) Patent Application Publication
(10) Pub. No.: US 2007/0173898 A1
(43) Pub. Date: Jul. 26, 2007

(54) Emergency controller for a medical device

(76) Inventors: Peter Joseph Ayre, Crows Nest (AU); Lee Thomas Glanzmann, Darlington (AU); Minh Lee, Kensington (AU); Nicholas Oliver Von Huben, Bexley North (AU)

Correspondence Address: DANIEL B. SCHEIN, PH.D., ESQ., INC., P. O. BOX 68128, Virginia Beach, VA, 23471, US

(21) Appl. No.: 11/643,649

(22) Filed: Dec. 20, 2006

(30) Foreign Application Priority Data
Dec. 20, 2005 (AU) 2005907171

(51) Int. Cl. A61N 1/00

(52) U.S. Cl. 607/33

(57) ABSTRACT

An emergency controller for use with a medical device adapted to be controlled by a primary controller. The emergency controller including a control unit, a power source, a connector and a switch, and wherein the connector is adapted to allow the emergency controller to be connected to the medical device or to the primary controller, and wherein the switch is adapted to activate the control unit when the emergency controller is connected to the medical device.

### FIELD OF THE INVENTION

The present invention relates to an emergency controller or control system for a medical device. Preferably, the medical device is an active implantable medical device.

### BACKGROUND OF THE INVENTION

Previously, there have been many control systems designed and manufactured suitable for use in relation to various medical devices (herein referred to as ‘MD’) and active implantable medical devices (herein referred to as ‘AIMD’). An AIMD may generally include a power source (e.g. a battery), control means (e.g. an electronic circuit) and a means providing the therapeutic action (e.g. an electrode or mechanical pump). AIMD typically include: rotary blood pumps, pacemakers, neural simulation implants, hearing aids and cochlear implants.

Previous control systems and/or controllers for AIMDs have relied on a single unit commonly known as a controller to actively control the operation and function of the AIMD. Typically, these controllers have been powered by a power source or a battery, which supplies electrical energy to the AIMD. Generally, the controllers may receive input data from sensors or derived data and use this data to feedback and provide operating parameters for the AIMD.

A disadvantage with this typical configuration is that the AIMD relies entirely on the functionality of the controller. In situations where the controller fails or operates improperly, a patient or clinician must exchange the faulty controller with a correctly functioning controller. The possibility of controller failure may mean that implanted patients usually are forced to carry with them, at all times, redundant controllers to be used in case of controller failure. This redundancy may add considerably to the overall bulk of the overall system and control system which the patient must carry. Additionally, patients must also remember the carry the replacement or redundant controllers with them at all times and this is difficult to achieve, particularly in elderly patients.

The present invention aims to or at least address or ameliorate one or more of the disadvantages associated with the above mentioned prior art.

### SUMMARY OF THE INVENTION

In accordance with a first aspect the present invention consists an emergency controller for use with a medical device adapted to be controlled by a primary controller, said emergency controller including a control unit, a power source, a connector and a switch, and wherein said connector is adapted to allow said emergency controller to be connected to said medical device or to primary controller, and wherein said switch is adapted to activate said control unit when said emergency controller is connected to said medical device.

Preferably, said switch is operated by the connection of a loop circuit included within at least one connector.

Preferably, said control unit, power source connector and switch are encapsulated within one housing.

Preferably, said switch deactivates said control unit when said emergency controller is connected to primary controller.

In accordance with a second aspect the present invention consists a control system for use with a medical device adapted to be controlled by a primary controller, said emergency controller including a control unit, a power source, a connector and a switch, and wherein said connector is adapted to allow said emergency controller to be connected to said medical device or to primary controller, and wherein said switch is adapted to activate said control unit when said emergency controller is connected to said medical device.

### BRIEF DESCRIPTION OF THE DRAWINGS

Embodiments of the present invention will now be described with reference to the accompanying drawings wherein:

FIG. 1 depicts a schematic representation of a first embodiment of the present invention;

FIG. 2 depicts a cross sectional view of a portion of the first embodiment as depicted in FIG. 1;

FIG. 3 depicts a cross sectional view of a portion of the first embodiment as depicted in FIG. 4;

FIG. 4 depicts a further schematic representation of the first embodiment; and

FIG. 5 depicts a diagrammatic view of a second embodiment of the present invention.

### BREIF DESCRIPTION OF THE PREFERRED EMBODIMENTS

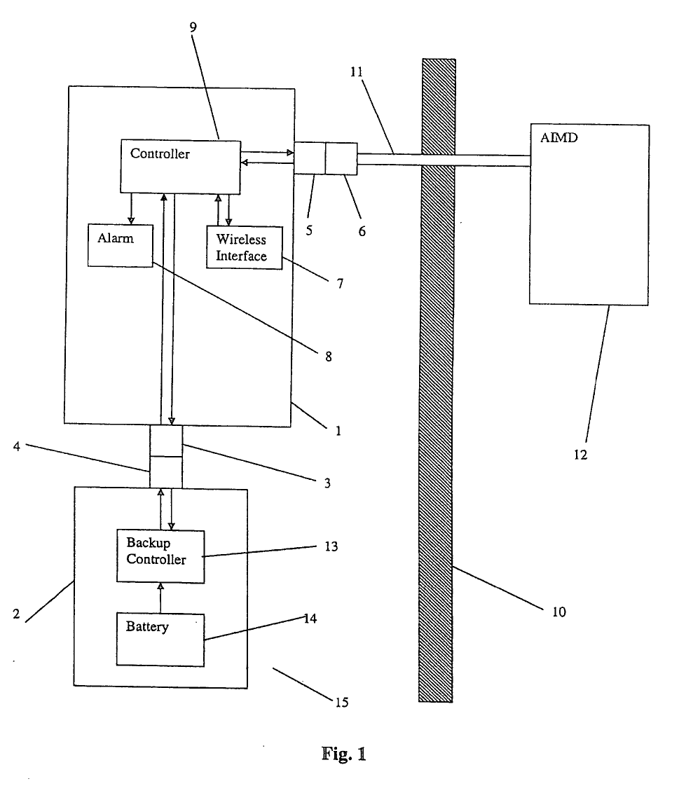

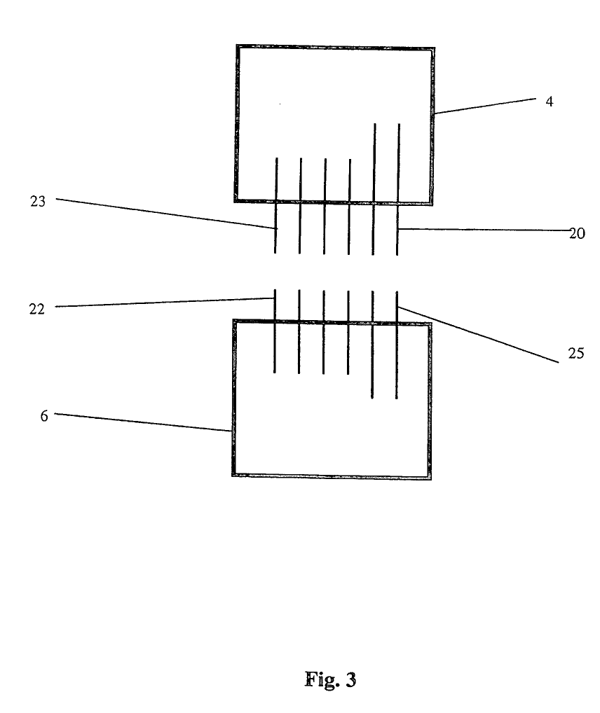

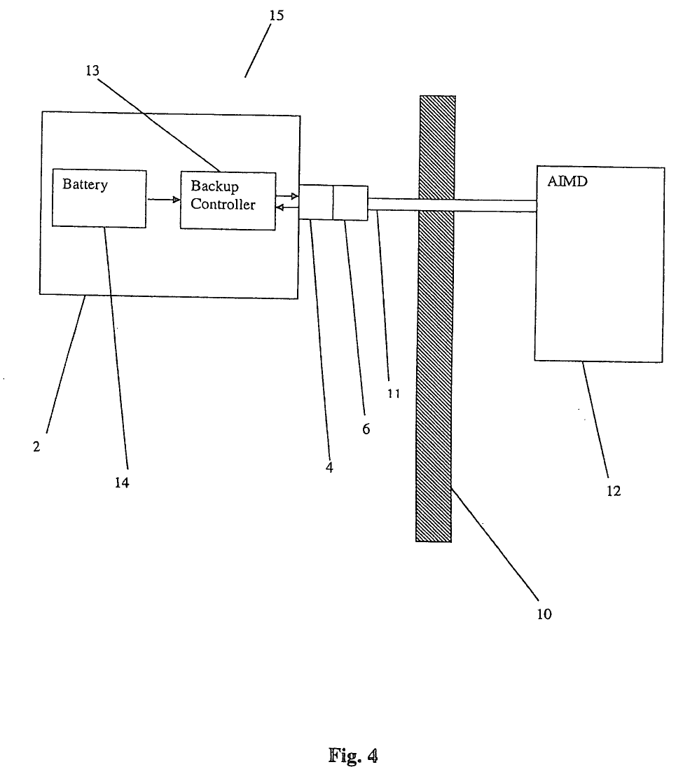

In a first embodiment of the present invention depicted in FIGS. 1 to 4, a control system 15 for use with an AIMD 12 is provided. The control system 15 comprises: first housing 1; second housing 2; wherein the first housing 1 includes: a primary controller 9, an alarm 8, and a wireless interface 7. In this first embodiment, the second housing 2 comprises an emergency control unit for use with an AIMD. Preferably, the emergency control unit within the second housing 2 includes: a backup controller 13 and at least one battery 14.

The primary controller 9, as depicted in FIG. 1 receives power from the battery 14 which is electrically connected by a first and second connectors 4 and 3. Preferably, the primary controller 9, as depicted in FIG. 1, controls and instructs the AIMD to operate and function within desired parameters. The primary controller 9 receives and transmits data and information relating to the operation of AIMD and the patient to external computers and/or software via a wireless interface 7. Preferably, the primary controller 9 also receives data and information from either the wireless interface 7 and/or the AIMD 12 and the primary controller 9 uses this information in deriving a preferred operating speed or function of the AIMD 12.

The primary controller 9 is preferably connected to the AIMD 12 by way of a percutaneous lead 11 that extends through the skin layer 10 of the patient. The primary controller 9 is electrically connected to the AIMD 12 via the use of a third and fourth connectors 5 and 6.

The second housing 2 includes a backup controller 13. In normal operating conditions and situations as depicted by FIG. 1, the backup controller 13 is inactive. However, if the primary controller 9 fails or functions improperly an alarm 8 is activated. The patient or clinician then may disengage the first housing 1 by the disconnection of the first and second connectors 3 and 4; and the disconnection of the third and fourth connectors 5 and 6 and thereby removing the first housing 1. The patient or clinician may then directly connect the second housing 2 to the AIMD 12 via the engagement of first and fourth connectors 4 and 6. When this engagement occurs the backup controller 13 automatically activates and may control the AIMD in a similar manner as to the primary controller 9.

The main advantage with this embodiment is that the backup controller 13 is miniaturised and fits within a part of the system that the patient must ordinarily carry with them namely, the second housing which encapsulates the battery 14. The patient is not required to carry a replacement primary controller with them. The backup controller 13 preferably is will function reliably and long enough for the patient to seek or obtain a replacement first housing 1.

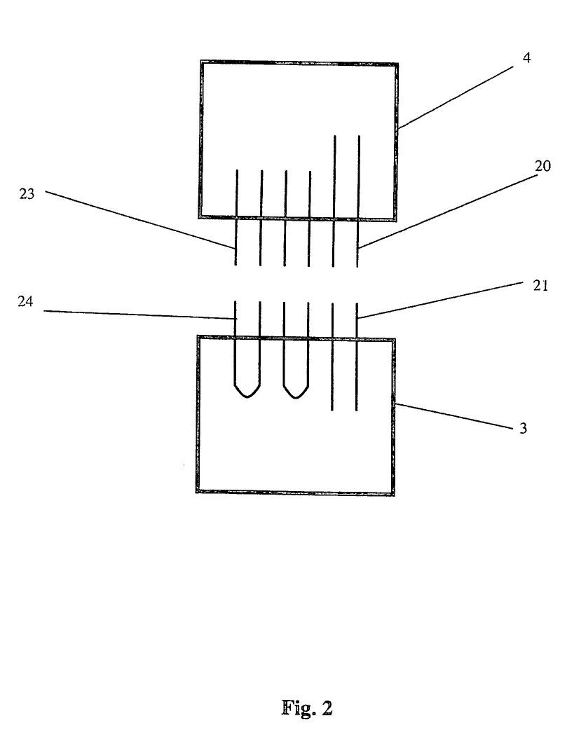

Preferably, the activation of the backup controller 13 is achieved by a switch mechanism preferably present within the system of connectors 3, 4, 5, and 6. As demonstrated in FIG. 2, the first connector 4 and the second connector 5 are connected in a configuration of FIG. 1. The primary pins 20 may supply power to the first housing 1 through the connection of the secondary pins 21. The first data pins 23 of first connector 4 preferably compromise four pins 23 which are capable of supplying and receiving instructions, data, and information to and from the backup controller 13, when activated. However, when the first set of data pins 23 is connected the second set of data pins 24, a loop circuit is formed, the backup controller 13 is then switched off by the connection of the loop circuit.

When the second housing 2 is connected directly to the AIMD 12 and the first housing 1 is removed from the control system 15. The first connector 4 engages the fourth connector 6. The first set of power pins 20 connect directly to the third set of power pins 25 which in turn directly powers the AIMD 12. The first set of data pins 23 are connected to the third set of data pins 22. As backup controller 13 does not detect a loop circuit, as shown in FIG. 2, the backup controller 13 activates and begins controlling the AIMD 12. Thereby the backup controller 13 is activated and deactivated by a switching mechanism present in the connectors.

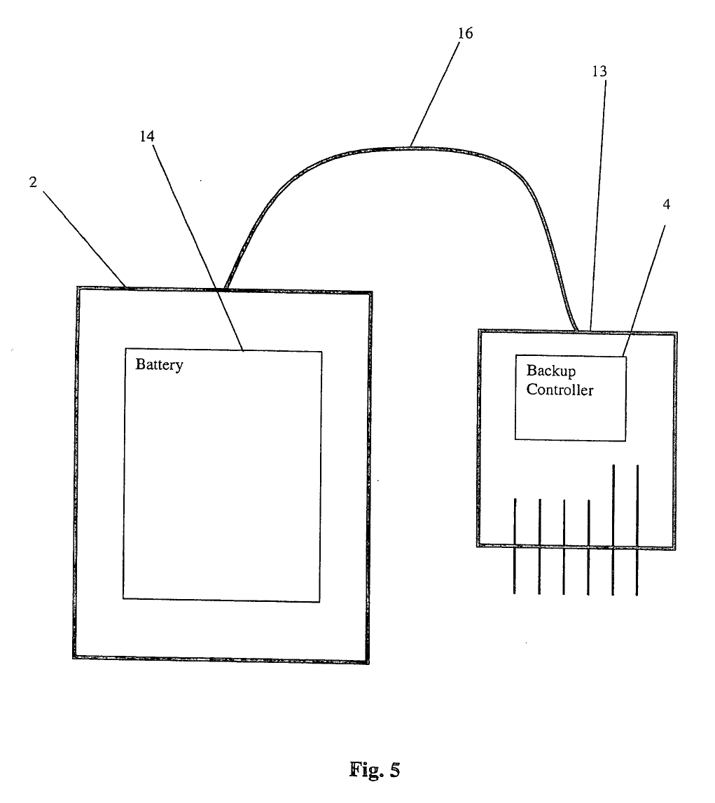

In a second embodiment of the present invention as depicted in FIG. 5, the backup controller 13 is encapsulated within the first connector 4 and the second housing 2 encapsulates the battery 14. This configuration may reduce the size and bulk of the overall control system 15 as the backup controller 13 has been further miniaturised to fit within the first connector 4. Preferably, the first connector 4 and the second housing 2 are electrically connected by a multi-stranded cable 16. Otherwise, the second embodiment is similar to the first embodiment depicted in FIGS. 1 to 7.

The preferred AIMD for use with this control system 15 is a left ventricular assist device in the form of a rotary blood pump. The preferred rotary blood pump is described in detail U.S. Pat. No. 6,227,797—Watterson et al and that disclosure is herein included within the present specification for the present invention.

The wireless interface 7 may be any standard wireless interface or protocol including, but not limited to: Bluetooth™, Zigbee™, Wi-Fi™ or 802.11a-g.

The percutaneous lead 11, which extends through the skin layer 10 may be replaced with transcutaneous energy transmission system (herein referred to as ‘TETS). TETS (not shown) generally may comprise of two electrical conductive coils positioned in parallel on either side of the skin layer 10. The two coils cooperate to transmit and receive RF energy and thereby power the AIMD 12. The advantage of TETS is that a permanent exit wound for the percutaneous lead 11 through the skin layer 10 is not required and thereby reducing the risk of infection or serious adverse events for an implanted patient.

The above descriptions detail only some of the embodiments of the present invention. Modifications may be obvious to those skilled in the art and may be made without departing from the scope and spirit of the present invention.

1. An emergency controller for use with a medical device adapted to be controlled by a primary controller, said emergency controller including a control unit, a power source, a connector and a switch, and wherein said connector is adapted to allow said emergency controller to be connected to said medical device or to primary controller, and wherein said switch is adapted to activate said control unit when said emergency controller is connected to said medical device.

2. The emergency controller as claimed in claim 1, wherein said switch is operated by the connection of a loop circuit included within at least one connector.

3. The emergency controller as claimed in claim 2, wherein said control unit, power source connector and switch are encapsulated within one housing.

4. The emergency controller as claimed in claim 1, wherein said switch deactivates said control unit when said emergency controller is connected to primary controller.

5. A control system for use with a medical device adapted to be controlled by a primary controller, said emergency controller including a control unit, a power source, a connector and a switch, and wherein said connector is adapted to allow said emergency controller to be connected to said medical device or to primary controller, and wherein said switch is adapted to activate said control unit when said emergency controller is connected to said medical device.

6. A power pack for use with a medical device adapted to be normally controlled by a primary controller connected to a first connector on said medical device, said power pack adapted to normally be connected to a second connector on said primary controller and supply power to both said primary controller and said medical device, and wherein said power pack comprises a power source, an emergency controller and a switch, and when said primary controller is disconnected from said medical device said power pack may be disconnected from said primary controller and connected directly to said medical device, said switch adapted to activate said emergency controller by disconnection of said power pack from said primary controller and/or by connection of said power pack to said medical device.
